(19) United States
(12) Patent Application Publication
(10) Pub. No.: US 2025/0230258 A1
(43) Pub. Date: Jul. 17, 2025

(54) ANTIBODY FOR PREDICTING BREAST CANCER PROGNOSIS AND NUCLEIC ACID ENCODING SEQUENCE THEREOF, AND USES OF THE SAME

(71) Applicants: National Tsing Hua University, Hsinchu (TW); CHANG GUNG MEMORIAL HOSPITAL, LINKOU, TAOYUAN CITY (TW)

(72) Inventors: Wen-Ching Wang, Hsinchu (TW); Wen-Ling Kuo, TAOYUAN CITY (TW)

(21) Appl. No.: 18/654,556

(22) Filed: May 3, 2024

(30) Foreign Application Priority Data
Jan. 12, 2024 (TW) 113101438

(51) Int. Cl. C07K 16/40; G01N 33/574

(52) U.S. Cl. CPC C07K 16/40; G01N 33/57415; C07K 2317/565; G01N 2474/20

(57) ABSTRACT

The present disclosure provides an antibody for predicting breast cancer prognosis and a nucleic acid encoding sequence thereof, and uses of the same. The antibody for predicting breast cancer prognosis of the present disclosure achieves the effect of predicting breast cancer prognosis through various efficacy experiments.

### CROSS-REFERENCE TO RELATED APPLICATION

This application claims priority of Taiwan patent application No. 113101438, filed on Jan. 12, 2024, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 112F0465-IE_Sequence_listing. The XML file is 9000 bytes; was created on Jan. 15, 2024.

### BACKGROUND OF THE INVENTION

### 1. Field of the Invention

The present invention relates to an antibody for predicting breast cancer prognosis and a nucleic acid encoding sequence thereof, and uses of the same.

### 2. The Prior Art

Breast cancer is a highly heterogeneous tumor and can be classified clinically based on its molecular characteristics. Different types of breast cancer have different tumor progression, treatment methods, recurrence and prognosis. How to effectively determine the risk of breast cancer recurrence and prognosis to select the appropriate treatment is an important clinical topic.

The early Nottingham prognostic index (NPI) and the later developed clinical treatment score post-5 years (CTS5) are all based on clinical characteristics as the criteria for determining prognosis. However, studies have reported that the prediction accuracy of CTS5 is lower in patients aged younger than 50 years.

Several genetic examining methods are used to evaluate prognosis in a patient with breast cancer and the sensitivity to chemotherapy, such as OncotypeDx, MammaPrint, EndoPredict, Prosigna, and breast cancer index (BCI). However, these methods are all based on the analysis of ribonucleic acid (RNA) expressions for a specific gene, and may not truly reflect the actual expressions of protein markers. It is impossible to provide clear mechanical instructions for the treatment of drugs.

In order to solve the above-mentioned problems, those skilled in the art urgently need to develop a novel method and medicament for predicting breast cancer prognosis for the benefit of a large group of people in need thereof.

### SUMMARY OF THE INVENTION

A primary objective of the present invention is to provide an antibody for predicting breast cancer prognosis, which specifically binds to pyruvate kinase isoenzyme M2 (PKM2), the antibody comprising a plurality of amino acid sequences, wherein each of the plurality of amino acid sequences is complementarity determining region (CDR), and the CDR comprises 5-12 amino acid residues.

Another objective of the present invention is to provide an isolated nucleic acid, encoding the above mentioned antibody.

Another objective of the present invention is to provide a pharmaceutical composition for predicting breast cancer prognosis, comprising the above mentioned antibody and a pharmaceutically acceptable carrier.

Another objective of the present invention is to provide a method for predicting breast cancer prognosis, comprising administering to a sample to be tested the above mentioned antibody, followed by interpretation of staining results by immunohistochemistry (IHC).

According to an embodiment of the present invention, the antibody specifically binds to O-linked β-N-acetylglucosamine (O-GlcNAc) on the PKM2.

According to an embodiment of the present invention, the antibody exhibits an antibody form selected from the group consisting of: single-chain variable fragment (scFv), minibody, nanobody, monoclonal antibody, immunoglobulin G (IgG), immunoglobulin A (IgA), and conjugated form.

According to an embodiment of the present invention, the plurality of amino acid sequences are as follows: first amino acid sequence, consisting of KSSQSLLYX₁NGK; second amino acid sequence, consisting of SEQ ID NO:2; third amino acid sequence, consisting of SEQ ID NO:3; fourth amino acid sequence, consisting of SEQ ID NO:4; fifth amino acid sequence, consisting of SEQ ID NO:5; and sixth amino acid sequence, consisting of MDGX₂YSLFDY; wherein X₁ is a neutral or hydrophilic amino acid residue, and X₂ is a hydrophobic amino acid residue.

According to an embodiment of the present invention, X₁ is selected from the group consisting of: aspartic acid (D), asparagine (N), glutamine (Q), serine (S), threonine (T), and glutamic acid (E).

According to an embodiment of the present invention, X₂ is selected from the group consisting of: methionine (M), alanine (A), valine (V), isoleucine (I), leucine (L), phenylalanine (F), tyrosine (Y), and tryptophan (W).

According to an embodiment of the present invention, the sample to be tested is a tissue section.

In summary, the antibody for predicting breast cancer prognosis of the present invention has the effect on directly identifying the O-GlcNAc protein modification on PKM2 in breast cancer specimens. Prognostic determination can be made with just one staining step, which greatly reduces the cost of testing and is cheaper than previously used gene expression testing methods such as OncotypeDx and MammaPrint. Additionally, in preliminary clinical data analysis, the antibody effectively predicts overall survival (OS) and disease-free survival (DFS) in breast cancer patients. This means that the antibody not only provides an accurate prognostic assessment, but also has a low cost of testing, making it an affordable breast cancer diagnostic tool. Therefore, the antibody of the present invention has the following advantages: 1. Specific recognition of specific molecular marker features: The antibody can specifically recognize specific molecular marker features in breast cancer tissue, such as O-GlcNAc protein modification on PKM2. This specific recognition makes the antibody a reliable tool for predicting the prognosis of breast cancer patients. 2. Highly accurate prognosis prediction: By staining and scoring tissue sections of breast cancer patients, the antibody can provide highly accurate prognosis prediction results. This helps doctors more accurately assess a patient's prognosis and make more informed decisions about individualized treatment. 3. Simplification and cost savings: Compared with traditional multiplex staining methods, the use of this antibody requires only one staining step, thus saving time, labor and material costs. This simplified detection process helps improve efficiency and reduce the cost of detection, making the technology more practical and feasible, and making it less expensive than multigene expression assays such as OncotypeDx and MammaPrint. 4. Application to personalized treatment strategies: The prognostic prediction results of this antibody can provide important reference for personalized treatment strategies for breast cancer patients. By understanding the patient's prognosis, doctors can better select and adjust treatment options to achieve the best clinical results. 5. Potential industrial application value: The application of this technology has potential value in the fields of biomedical testing, clinical diagnosis and pharmaceuticals. Developing related industry products, such as breast cancer diagnostic kits, clinical diagnostic services and drug research and development, can help improve breast cancer diagnosis.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIGS. 1A-1D show characterization of O-GlcNAcylated PKM2. (1A) O-GlcNAcylation of PKM2 but not PKM1. Recombinant His-tagged PKM2 or His-tagged PKM1 was incubated with O-GlcNAc transferase (OGT) and UDP-GlcNAc, followed by Western blot analysis using anti-O-GlcNAc and anti-PKM antibodies (as indicated). (1B) Analysis of bacterial His-tagged PKM2 and eukaryotic Flag-tagged PKM2. Bacterial recombinant PKM2 was purified in E. coli transformed with the respective expression vector using a Ni-NTA affinity column (left). Flag-tagged PKM2 proteins were obtained in HEK293 cells transfected with the respective expression vector (right). (1C) LC-MS/MS analysis of O-GlcNAcylated PKM2 from (1B) identified G-TX as the most susceptible O-GlcNAcylation site. (1D) The PKM2 monomer structure is shown as a cartoon, and the potential O-GlcNAc glycosylation modification site (TX/T405/S406) is shown as a stick.

FIGS. 2A-2F show characterization of monoclonal antibodies targeting O-GlcNAcylated PKM2. (2A) Specificity of monoclonal antibodies against O-GlcNAcylated PKM2. Recombinant wild-type (WT) and O-GlcNAcylated PKM2 variant 4A (T405AS406AT409AT454A) proteins were prepared (left panel). An ELISA assay was conducted to assess the specificity of four monoclonal antibody clones (2-3D, 3-8E, 5-5G, and 5-7D) towards O-GlcNAcylated PKM2 (G-4A) compared to WT (right panel). (2B) Specificity of 5-7D mAb demonstrated by dot blot assay. Various quantities of biotinylated-PKM2, biotinylated-O-GlcNAcylated TX PKM2, and KLH-O-GlcNAcylated TX PKM2 peptides were applied to a nitrocellulose membrane and subjected to immunoblotting using 5-7D mAb. (2C-2D) Interaction of 5-7D mAb with O-GlcNAcylated TX PKM2 peptide (2C) or TX PKM2 peptide (2D) evaluated by Elisa assay. (2E) Formation of complexes between 5-7D mAb and O-GlcNAcylated PKM2 analyzed by size exclusion chromatography. Recombinant PKM2 proteins were incubated with or without 5-7D mAb, followed by size exclusion chromatography. Immunoblotting was performed using anti-PKM2, anti-O-GlcNAcylated, and HRP-conjugated anti-mouse IgG antibodies. (2F) Immunoprecipitation assay with 5-7D mAb followed by Western blot analysis. Wild-type (WT) PKM2, O-GlcNAcylated WT (G-WT), PKM2 TXA variant (TXA), and O-GlcNAcylated TXA (G-TXA) were used in the assay.

FIGS. 3A and 3B show immunohistochemical analysis of 5-7D staining and its prognostic indications in luminal breast cancer patients. (3A) Representative immunohistochemistry (IHC) images demonstrating 5-7D staining. Magnification: 100× (bottom right: 200×). (3B) Strong immunostaining of 5-7D is associated with decreased overall survival (OS) and disease-free survival (DFS) rates. The IHC Q score was calculated based on the intensity and positive proportion scores. Samples were categorized as Low (Q≤mean) or High (Q>mean).

FIG. 4 shows logistic regression and receiver operating characteristic (ROC) curve analyses.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, clinical treatment score post-5 years (CTS5) is a calculation tool integrating four clinical data including tumor size, tumor grade, patient age, and the number of nodes to estimate the risk of breast cancer.

According to the present invention, the pharmaceutical composition can be manufactured to a dosage form suitable for parenteral administration, using techniques well known to those skilled in the art, including, but not limited to, injection (e.g., sterile aqueous solution or dispersion), sterile powder, dispersible powder or granule, solution, suspension, emulsion, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

As used herein, the term “nucleic acid”, “nucleic acid sequence” or “nucleic acid fragment” refers to a sequence of deoxyribonucleotides or ribonucleotides in single- or double-stranded forms, and comprises known naturally occurring nucleotides or artificially chemical mimics. As used herein, the term “nucleic acid” is used interchangeably with the terms “gene”, “cDNA”, “mRNA”, “oligonucleotide” and “polynucleotide”.

According to the present invention, the procedure regarding subjects is as follows. All subjects were recruited from Chang-Gung Memorial Hospital (CGMH), Linkou, Taiwan (2005-2013). Demographic, pathological, and clinical data included age, history of diabetes, estrogen receptor (ER) and progesterone receptor (PR) status, tumor size, grade, tumor node (TN) stage, surgical treatment, chemotherapy use, endocrine therapy, and radiotherapy for each subject. Among them, 154 early breast cancer patients who had received standard treatment and routine follow-ups for recurrence status since 2007 were recruited: 49 patients with documented recurrence and 105 patients without any recurrence over 10 years. The study protocol was approved by the Institutional Review Board of CGMH (IRB #201700716A3).

According to the present invention, the procedure regarding plasmid construction is as follows. Pyruvate kinase isoenzyme M2 (PKM2) full-length cDNA was ligated to the pET28a (Novagen) vector for E. coli expression. All PKM2 mutants (WT, TXA, 4A (T405AS406AT409AT454A)) were generated using the site-directed mutagenesis method. For eukaryotic cell expression, PKM2 wild type and mutant cDNAs were inserted into the pcDNA3.1 vector. All sequences were verified by DNA sequencing.

According to the present invention, the procedure regarding purification of recombinant PKM2 is as follows. Protein expression of E. coli BL21 (DE3) carrying PKM2 wild type/mutant plasmids was induced by 1.0 mM IPTG (isopropyl-β-D-thiogalactopyranoside) at 16° C. To obtain bacterial O-GlcNAcylated PKM2, recombinant His-tagged PKM2 and GST-tagged O-GlcNAc transferase (OGT) were co-expressed in Escherichia coli. Wild type or mutant PKM2 was cloned into the pET28a vector, and OGT was cloned into the pGEX-4t-1 vector. Both vectors were co-transformed into E. coli BL21(DE3) competent cells. Single colonies expressing both proteins were confirmed by small-scale expression tests. For the expression of mammalian O-GlcNAcylated PKM2, HEK293T cells were co-transfected with Flag-tagged PKM2 and GFP-tagged OGT, followed by immunoprecipitation.

For large-scale purification, a 10-mL overnight culture of the bacterial strain in LB broth supplemented with 35 μg/mL kanamycin and 100 μg/mL ampicillin was scaled up to a 1 L LB culture. After 5 hours of incubation at 37° C. and 180 rpm, protein expression was induced by 0.5 mM IPTG at 16° C. and 180 rpm for 21 hours. The bacteria were then harvested by centrifugation at 6000×g and 4° C. for 20 minutes. The cell pellets were lysed, and the O-GlcNAcylated PKM2 protein was purified using a Ni-NTA affinity column and size-exclusion chromatography.

Cells were harvested and homogenized by sonication. After centrifugation (10,000×g at 4° C. for 20 min), the recombinant protein was purified from the crude extract using cobalt-chelated TALON Metal Affinity Resin (Clontech) following the manufacturer's instructions. The eluted protein was concentrated and dialyzed into Tris buffer (40 mM Tris, 100 mM KCl, pH 7.5) using an Amicon Ultra-15 30,000 MW tube (Millipore). In the next purification step, the concentrated PKM2 was subjected to lectin-based affinity purification using succinylated wheat germ agglutinin (sWGA) resin (Vector Laboratories) to enrich glycosylated PKM2. The protein was eluted from the resin using buffer A supplemented with 0.5 M N-acetyl-D-glucosamine. The eluate was concentrated and dialyzed using a centrifugal filter (Amicon® Ultra-15 30K, Millipore). The purified protein was analyzed by sodium dodecyl sulfate-polyacrylamide gel electrophoresis (SDS-PAGE) and Coomassie brilliant blue staining. Protein purity was checked by SDS-PAGE followed by Coomassie brilliant blue staining.

According to the present invention, the procedure regarding measurement of PKM2 activity is as follows. In vitro PKM2 pyruvate kinase activity was determined by measuring the OD340 of the lactate dehydrogenase-coupled reaction. A steady-state kinetic reaction was carried out in 50 mM Tris (pH 7.5), 100 mM KCl, 5 mM MgCl₂, 1 mM ADP, 0.4 mM NADH, 2 U LDH, 25 ng PKM2, and the [phosphoenolpyrivate (PEP)] in the range of 0.06 to 8 mM in a total volume of 200 μL at 37° C.

According to the present invention, the procedure regarding size-exclusion chromatography is as follows. Size-exclusion chromatography was performed using an ÄKTA FPLC system. The PKM2 protein was aliquoted to 100 μL at a concentration of 2.5 mg/mL in PBS. The protein was then run through a Bio-Rad ENrich™ SEC 650 column, equilibrated with PBS, at a flow rate of 1 mL/min. The elution process was monitored by measuring OD280, and the eluted protein was collected in 0.5 mL fractions.

According to the present invention, the procedure regarding LC-MS/MS analysis and data processing for mapping O-GlcNAcylation sites of PKM2 is as follows. In-gel digestion: For in-gel trypsin digestion, the ˜70 kDa protein bands from an SDS-PAGE gel were excised into ˜1 mm cubes and subjected to in-gel digestion. The excised gel pieces were destained by repeated washes with 40% acetonitrile (ACN)/25 mM ammonium bicarbonate, followed by reduction with 10 mM dithioerythritol for 1 h at 37° C. and alkylation with 55 mM iodoacetamide at room temperature in the dark for 1 h. Subsequently, the gel slices were dried and digested first with Lys-C protease at 37° C. for 3 h, followed by trypsin digestion at 37° C. overnight. The resulting tryptic peptides were extracted twice with 50% ACN/5% trifluoroacetic acid and once with ACN. The extracts from each sample were combined and dried using a SpeedVac. After digestion, the peptide mixtures were desalted using C18 StageTips (Rappsilber, Mann et al. 2007) and resuspended in 0.1% formic acid (FA) for LC-MS/MS analysis.

Mass spectrometry to map the O-GlcNAcylation sites of PKM2: LC-MS/MS analysis was conducted on an EASY-nLC™ 1200 system connected to a Thermo Orbitrap Fusion™ Lumos™ Tribrid™ Mass Spectrometer (Thermo Fisher Scientific, Bremen, Germany) via a Nanospray Flex™ ion source (Thermo Fisher Scientific, Bremen, Germany). Peptide mixtures were loaded onto a PepMap C18 column (75 μm ID, 25 cm length, 2 μm particles, pore size 100 Å, Thermo Fisher Scientific) and separated using a 55-minute segmented gradient from 6% to 50% solvent B (80% ACN with 0.1% FA) at a flow rate of 300 nl/min. Solvent A was 0.1% FA in water. The mass spectrometer was operated in data-dependent mode. Survey scans of peptide precursors ranging from 350 to 1800 m/z were performed at 120K resolution with a 2×10\hat{ }5 ion count target. The Top Speed method was enabled to ensure full MS spectra were acquired every 3 s. Precursor ions with 2-8 charges and ion count above a threshold of 50,000 were selected for data-dependent higher energy collision dissociation (HCD) at a resolution of 30K and a normalized collision energy of 30%. If peaks at m/z 138.0545 (HexNAc oxonium fragment ions) or m/z 204.0867 (HexNAc oxonium ions) were detected within the top 50 most abundant peaks, a subsequent electron-transfer/higher energy collision dissociation (EThcD) MS/MS scan of the precursor ion was triggered and acquired in the Orbitrap at a resolution of 30K.

Data processing: All MS and MS/MS raw spectra from each sample were searched against the Human PKM2 protein sequence using Proteome Discoverer 2.1 (Thermo Fisher Scientific, San Jose, CA) with the Byonic v. 2.16.11 (Protein Metrics, San Carlos, CA) search engine. The peptide search parameters included two missed cleavages for full trypsin digestion, with fixed carbamidomethyl modification of cysteine, variable modifications of methionine oxidation, deamidation on asparagine or glutamine residues, and O-linked β-N-acetylhexosamine (O-GlcNAc) modification of serine and threonine. The peptide mass tolerance was 10 ppm and fragment mass tolerance values for HCD and EThcD spectra were 20 ppm.

According to the present invention, the procedure regarding immunization of mice is as follows. Eight-week-old Balb/c mice were immunized four times with 50 μg of purified O-GlcNAcylated PKM2 protein. The first immunization was emulsified with complete Freund's adjuvant (1:1 mixed with recombinant protein, Sigma-Aldrich, St. Louis, MO, USA), while the subsequent immunizations were emulsified with incomplete Freund's adjuvant. The mice were injected intraperitoneally on days 0, 7, 12, 19, and 29. Serum titers were measured by indirect ELISA three days after the third and fourth immunizations. Three days after the last booster immunization, the spleen of the mouse with the highest titer was removed under sterile conditions to prepare a splenic lymphocyte suspension for cell fusion.

According to the present invention, the procedure regarding cell culture and fusion is as follows. The mouse myeloma cell line FO was cultured in DMEM medium (Gibco-BRL, Grand Island, NY, USA) supplemented with 10% fetal bovine serum (Hyclone, Tauranga, New Zealand) at 37° C. and 5% CO₂. Splenic lymphocytes (2×10⁷) were prepared using the MACS® Spleen Dissociation Kit (Miltenyi Biotec, Bergisch Gladbach, Germany) and fused with FO cells (1×10⁷) using 50% polyethylene glycol 1500 (PEG 1500, Sigma-Aldrich, St. Louis, MO, USA). The hybridoma cells were cultured in 96-well plates with DMEM medium supplemented with 20% fetal bovine serum and hypoxanthine-aminopterin-thymidine (HAT; Sigma-Aldrich, St. Louis, MO, USA). After two weeks, indirect ELISA was performed to detect positive clones, using 96-well plates coated with 100 ng of purified PKM2 or O-GlcNAcylated PKM2-4A protein. Screened clones were re-cloned by limiting dilution in DMEM supplemented with 20% FBS and HAT for an additional 2 weeks.

According to the present invention, the procedure regarding screening of positive clones and cloning is as follows. A single clone was selected by observing the 96-well plates, and indirect ELISA was performed using 100 ng of purified PKM2 or O-GlcNAcylated PKM2-4A as coating proteins. Purified monoclonal antibody was serially diluted and incubated with the coated plates. After incubation with HRP-conjugated goat anti-mouse antibody, TMB substrate was used for color development. The optical density (OD) was measured at 650 nm using a microplate reader. Positive clones were identified as those with an OD test to OD control ratio greater than 1.5 times. Positive wells were then recloned by limiting dilution. The positive clones were transferred to 24-well plates, enlarged to 10 cm culture dishes in DMEM with 10% FBS, and a specific monoclonal antibody-secreting hybridoma cell strain was established.

According to the present invention, the procedure regarding antibody purification is as follows. The ascites method of anti-TX-O-GlcNAcylated PKM2 antibody production and purification was helped by LTK BioLaboratories (Taoyuan City, Taiwan).

According to the present invention, the procedure regarding Western blotting and dot-blot assay is as follows. 2 μg of recombinant protein or 20 μg of total proteins were separated by SDS-PAGE on a 10% polyacrylamide gel and then transferred onto an Amersham™ Protran™ nitrocellulose membrane (GE Healthcare Life Science, Washington, D.C., USA) by electroblotting. The membrane was blocked using 5% BSA at 37° C. for 30 minutes. Next, the membrane was incubated with the indicated primary antibody overnight at 4° C. Afterward, the membrane was washed with PBS-Tween-20 (PBST) three times and incubated with the HRP-conjugated goat anti-mouse secondary antibody (1:5,000) at 37° C. for 30 minutes with shaking. Following three washes with PBST, the membrane was visualized using Western Lightning® Plus-ECL (PerkinElmer, Inc., Waltham, MA, USA). For the dot plot assay, the purified anti-TX-O-GlcNAcylated PKM2 antibody was applied to a nitrocellulose membrane with relative O-GlcNAcylated peptides and immunoblotting analysis of purified PKM2 and TX-O-GlcNAcylated PKM2 protein in different dilution for titer determination and specificity analysis. The recombinant PKM2 (PKM2 UniProt accession number: P-14618-1), O-GlcNacylated PKM2, PKM2-TXA and O-GlcNacylated PKM2-TXA were used to immunoprecipitation with anti-TX-O-GlcNAcylated PKM2 antibody and analyzed with western blotting.

According to the present invention, the procedure regarding immunocytochemistry (IHC) is as follows. Paraffin-embedded sections were baked for 60 minutes and then soaked in xylene for 20 minutes to remove paraffin wax. Rehydration was performed using different concentrations of ethanol, and the sections were subjected to antigen retrieval with unmasking solution (0.01 M sodium citrate buffer with 0.05% Tween 20, pH 6.0) at just below boiling temperature for 10 minutes. After washing with TBST, the sections were stained with the indicated antibody and the BOND Polymer Refine Detection kit according to the manufacturer's instructions (Leica Biosystems, Wetzlar, Germany). Finally, the sections were dehydrated with different concentrations of ethanol and mounted with mounting medium. Images were captured using a Motic EasyScan One slide scanner (Meyer Instruments, Inc., Houston, TX, USA) and evaluated by a pathologist. IHC was performed for O-GlcNAc (838004, Biolegend, San Diego, CA, USA) and PKM2 (D78A4, Cell Signaling Technology, Danvers, MA, USA). IHC results were reviewed blindly by two pathologists (CJ Chen and YC Hsu) based on two parameters: staining intensity score (0=negative; 1=weak; 2=moderate; and 3=strong) and the percentage of immunopositive cells (0-100). The Q score (0-300) was calculated by multiplying these two parameters. Two groups were stratified: low expression (Q≤mean) and high expression (Q>mean). Kaplan-Meier analysis was conducted to evaluate clinical relevance.

According to the present invention, the procedure regarding statistical analysis is as follows. The Chi-square test was used to compare the clinicopathological properties of patients with categorical variables. Kaplan-Meier curve analysis was conducted to assess overall patient survival, defined as the time from diagnosis until death or the last follow-up. The log-rank test was employed to determine the statistical significance between groups. Logistic regression analysis was performed to assess the contribution of O-GlcNAc and PKM2 expression, and/or the CTS5 score in prognosis. Receiver operating characteristic (ROC) curves were plotted based on the optimal set of sensitivity and specificity. The ROC curves for each of the logistic models were generated using the fitted probabilities from the model as possible cut-points for computing sensitivity and specificity. The area under the curve (AUC) was calculated using numerical integration of the ROC curves. A p-value of less than 0.05 was considered significant. All p-values were 2-sided. Statistical analysis was performed using IBM SPSS, version 25 (IBM Corp.). For unadjusted comparisons, a p-value of <0.05 was considered statistically significant.

The antibody according to the present invention can comprise an amino acid sequence of SEQ ID NO:1-SEQ ID NO:6, wherein the amino acid sequence of SEQ ID NO:1-SEQ ID NO:6 is complementarity determining region (CDR). Preferably, the CDR comprises an amino acid sequence having at least 70% sequence similarity with the amino acid sequence of SEQ ID NO:1-SEQ ID NO:6.

The antibody according to the present invention can comprise an amino acid sequence of SEQ ID NO:7 and SEQ ID NO:8, wherein the amino acid sequence of SEQ ID NO:7 is the antibody light chain, and the amino acid sequence of SEQ ID NO:8 is the antibody heavy chain. The amino acid sequence of SEQ ID NO:1-SEQ ID NO:3 is also located on the antibody light chain, and the amino acid sequence of SEQ ID NO:4-SEQ ID NO:6 is also located on the antibody heavy chain. In particular, the aspartic acid (D) residue in the amino acid sequence of SEQ ID NO:1 can be replaced with a neutral or hydrophilic amino acid, and the methionine (M) residue at residue No. 4 of the amino acid sequence of SEQ ID NO:6 can be replaced with a hydrophobic amino acid. The neutral or hydrophilic amino acid can be asparagine (N), glutamine (Q), serine (S), threonine (T), or glutamic acid (E). The hydrophobic amino acid can be alanine (A), valine (V), isoleucine (I), leucine (L), phenylalanine (F), tyrosine (Y), or tryptophan (W).

### Example 1

### Characterization of O-GlcNAcylation Sites and Functional Implications on Pyruvate Kinase Isoenzyme M2 (PKM2)

To investigate the impact of O-GlcNAcylation on pyruvate kinase isoenzyme M2 (PKM2) in cancer diagnosis and prognosis, we employed both prokaryotic and eukaryotic expression systems to produce and purify recombinant O-GlcNAcylated PKM2. In the E. coli BL21(DE3) system, we co-expressed His-tagged PKM2/PKM1 and GST-tagged O-GlcNAc transferase (OGT), resulting in the generation of recombinant O-GlcNAcylated PKM2/PKM1 (PKM2 accession number: P-14618-1, PKM1 accession number: P14618-2).

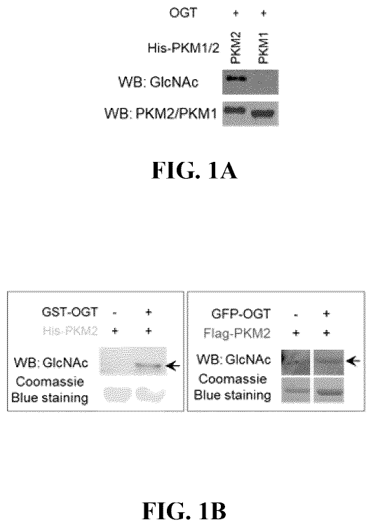

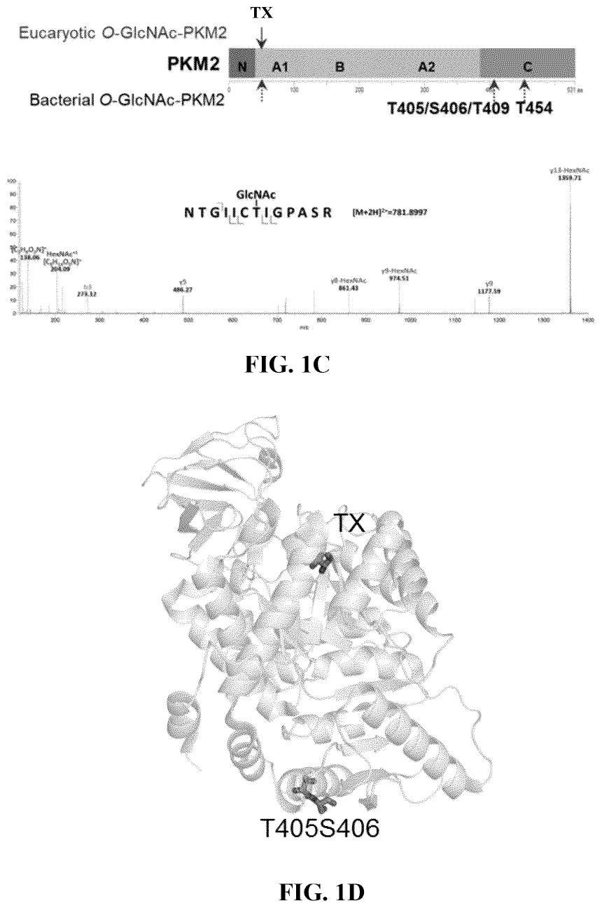

FIGS. 1A-ID show characterization of O-GlcNAcylated PKM2. (1A) O-GlcNAcylation of PKM2 but not PKM1. Recombinant His-tagged PKM2 or His-tagged PKM1 was incubated with O-GlcNAc transferase (OGT) and UDP-GlcNAc, followed by Western blot analysis using anti-O-GlcNAc and anti-PKM antibodies (as indicated). (1B) Analysis of bacterial His-tagged PKM2 and eukaryotic Flag-tagged PKM2. Bacterial recombinant PKM2 was purified in E. coli transformed with the respective expression vector using a Ni-NTA affinity column (left). Flag-tagged PKM2 proteins were obtained in HEK293 cells transfected with the respective expression vector (right). (1C) LC-MS/MS analysis of O-GlcNAcylated PKM2 from (1B) identified G-TX as the most susceptible O-GlcNAcylation site. (1D) The PKM2 monomer structure is shown as a cartoon, and the potential O-GlcNAc glycosylation modification site (TX/T405/S406) is shown as a stick.

Through purification using His-tag and glycan-affinity enrichment methods, we conducted Western blot analysis to examine O-GlcNAcylation, revealing the preferential modification of PKM2 by OGT compared to PKM1 (FIG. 1A).

To identify crucial O-GlcNAcylated sites on PKM2, we subjected purified bacterial His-tagged recombinant PKM2 or O-GlcNAcylated PKM2, which displayed prominent O-GlcNAcylated signals (FIG. 1B, left), to LC-MS/MS analysis. This analysis unveiled several O-GlcNAcylated sites, including TX, T405/S406/S409, and T454 (FIG. 1C). Further, eucaryotic Flag-tagged PKM2 and O-GlcNAcylated PKM2 were expressed and purified from HEK293T cells expressing Flag-tagged PKM2 or O-GlcNAcylated PKM2 (FIG. 1B, right). Immunoprecipitation and LC-MS/MS analysis confirmed the presence of O-GlcNAc only at the TX site (FIG. 1C), highlighting its susceptibility to O-GlcNAcylation in mammalian PKM2.

The O-GlcNAcylated residues T405 and S406, encoded by exon 10, were found to reside at the C-C interface of tetrameric PKM2 (FIG. 1D). Notably, T454, located at the junction between the A and C domains, was identified as a phosphorylation site for PIM2.

### Example 2

### Generation and Characterization of Monoclonal Antibodies Targeting O-GlcNAcylated PKM2

To investigate the potential use of O-GlcNAcylated PKM2 as a diagnostic and prognostic biomarker for breast cancer, we developed monoclonal antibodies (mAbs) against O-GlcNAcylated PKM2 using a hybridoma approach.

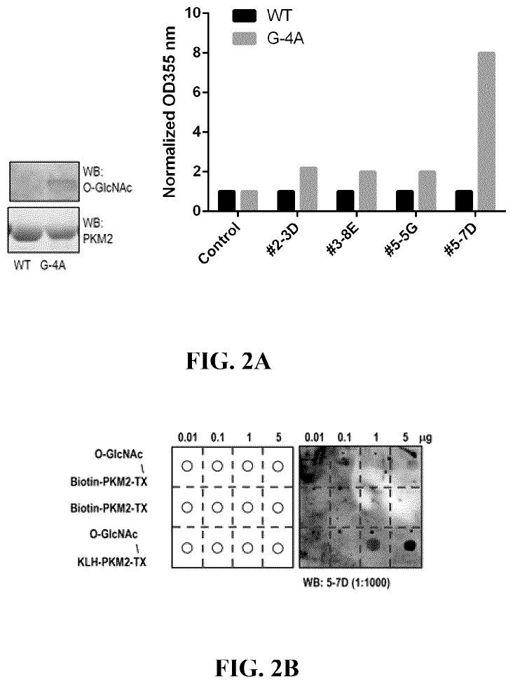

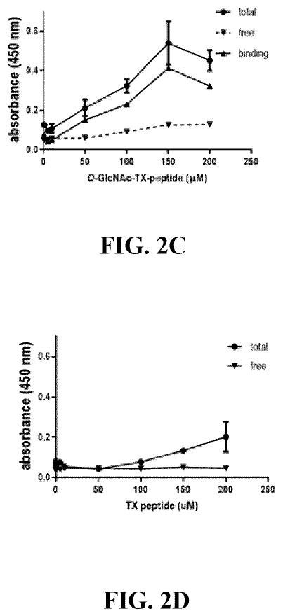

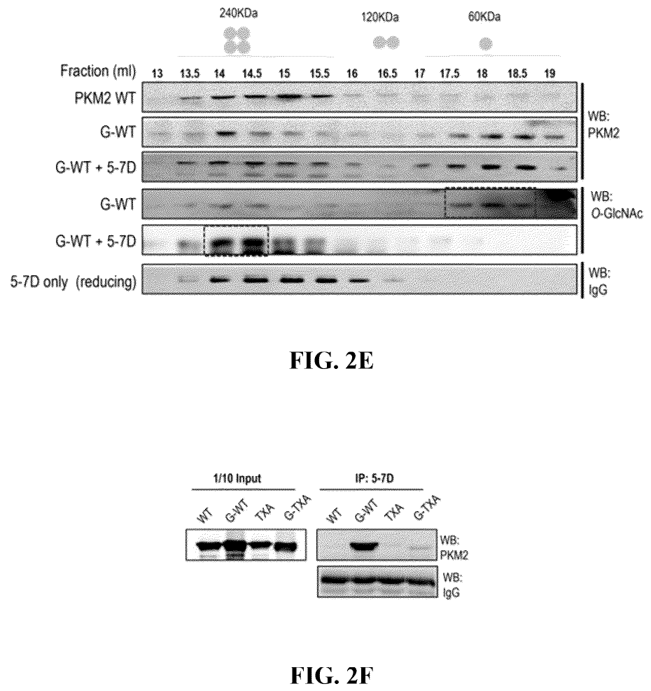

FIGS. 2A-2F show characterization of monoclonal antibodies targeting O-GlcNAcylated PKM2. (2A) Specificity of monoclonal antibodies against O-GlcNAcylated PKM2. Recombinant wild-type (WT) and O-GlcNAcylated PKM2 variant 4A (T405AS406AT409AT454A) proteins were prepared (left panel). An ELISA assay was conducted to assess the specificity of four monoclonal antibody clones (2-3D, 3-8E, 5-5G, and 5-7D) towards O-GlcNAcylated PKM2 (G-4A) compared to WT (right panel). (2B) Specificity of 5-7D mAb demonstrated by dot blot assay. Various quantities of biotinylated-PKM2, biotinylated-O-GlcNAcylated TX PKM2, and KLH-O-GlcNAcylated TX PKM2 peptides were applied to a nitrocellulose membrane and subjected to immunoblotting using 5-7D mAb. (2C-2D) Interaction of 5-7D mAb with O-GlcNAcylated TX PKM2 peptide (2C) or TX PKM2 peptide (2D) evaluated by Elisa assay. (2E) Formation of complexes between 5-7D mAb and O-GlcNAcylated PKM2 analyzed by size exclusion chromatography. Recombinant PKM2 proteins were incubated with or without 5-7D mAb, followed by size exclusion chromatography. Immunoblotting was performed using anti-PKM2, anti-O-GlcNAcylated, and HRP-conjugated anti-mouse IgG antibodies. (2F) Immunoprecipitation assay with 5-7D mAb followed by Western blot analysis. Wild-type (WT) PKM2, O-GlcNAcylated WT (G-WT), PKM2 TXA variant (TXA), and O-GlcNAcylated TXA (G-TXA) were used in the assay.

Out of 1248 clones screened, four independent clones (2-3D, 3-8E, 5-5G, and 5-7D) exhibited enhanced specificity towards the O-GlcNAcylated PKM2 variant 4A compared to the wild-type PKM2 (FIG. 2A). Further characterization of the 5-7D mAb, which displayed the highest specificity, revealed its selective interaction with the O-GlcNAcylated TX PKM2 peptide while showing no binding affinity to the PKM2 peptide (FIGS. 2B-D). Subsequently, we performed size exclusion chromatography to evaluate the complex formation between the 5-7D mAb and recombinant O-GlcNAcylated PKM2 protein or PKM2. Western blot analysis demonstrated that the PKM2 tetramer was detected at approximately 240 kDa (fractions 13.5-15.5), while the O-GlcNAcylated PKM2 predominantly existed as monomers (fractions 17-19). Interestingly, in the presence of the 5-7D mAb, the O-GlcNAcylated PKM2 exhibited significant shifts in higher molecular weight species within the O-GlcNAcylated PKM2/5-7D mixture (FIG. 2E). Furthermore, through a pull-down assay, we provided evidence supporting the selective binding of the 5-7D mAb to O-GlcNAcylated PKM2, as it formed complexes with recombinant O-GlcNAcylated PKM2 protein but not with recombinant O-GlcNAc TXA protein (FIG. 2F). Notably, immunofluorescence assays using O-GlcNAcylated TX peptides hindered the detection of the 5-7D mAb. Collectively, our findings highlight the high specificity of the 5-7D mAb in recognizing O-GlcNAcylation of PKM2 at TX, showcasing its potential for diagnostic and prognostic applications in breast cancer research.

In this example, the amino acid sequence of 5-7D mAb consists of the amino acid sequence of SEQ ID NO:7 and SEQ ID NO:8.

### Example 3

### Prognostic Value of 5-7D in Recurrent Luminal Breast Cancer

To evaluate the prognostic potential of 5-7D in recurrent luminal breast cancer, we conducted a retrospective study involving 154 patients who received breast cancer therapy at the Department of Surgery, Chang Gung Memorial Hospital Linkou Medical Center. The study cohort consisted of 105 non-recurrent patients (median age [interquartile range (IQR)]: 53.0 [18.0] years) and 49 recurrent patients (median age [IQR]: 51 [16] years), who were followed up until December 2018 (median follow-up period: 123.4 months, IQR: 107.4-134.1 months) (Table 1).

TABLE 1

| | | Non-recurrent | Recurrent | |
|---|---|---|---|---|
| | | No. (%), | No. (%), | p |
| Factors | | n = 105 | n = 49 | value |
| Molecular | Luminal A | 70 (66.7) | 35 (71.4) | 0.555 |
| subtype | Luminal B | 35 (33.3) | 14 (28.6) | |
| Age (years) | Median (IQR) | 51 (16) | 53 (21) | 0.533 |
| | ≤50 | 49 (46.7) | 20 (40.8) | 0.497 |
| | >50 | 56 (53.3) | 29 (59.2) | |
| Diabetes mellitus | No | 97 (92.4) | 44 (89.8) | 0.756 |
| | Yes | 8 (7.6) | 5 (10.2) | |
| Operation type | Mastectomy | 56 (53.3) | 27 (55.1) | 0.838 |
| | Breast | 49 (46.7) | 22 (44.9) | |
| | conservation | | | |
| Invasive tumor | Median (IQR) | 1.9 (1.4) | 2.4 (1.7) | 0.022 |
| size (cm) | | | | |
| SBR grade^{a} | 1 | 13 (12.4) | 5 (10.2) | 0.720 |
| | 2 | 57 (54.3) | 30 (61.2) | |
| | 3 | 35 (33.3) | 14 (28.6) | |
| Estrogen receptor | Negative | 4 (3.8) | 0 | 0.307 |
| | Positive | 101 (96.2) | 49 (100.0) | |
| Progesterone | Negative | 10 (9.5) | 3 (6.1) | 0.553 |
| receptor | Positive | 95 (90.5) | 46 (93.9) | |
| Ki67 index | <25 | 26 (24.8) | 16 (32.7) | 0.003 |
| | ≥25 | 14 (13.3) | 16 (32.7) | |
| | Missing | 65 (61.9) | 17 (34.6) | |
| T stage | T1a | 0 | 1 (2.0) | 0.023 |
| | T1b | 7 (6.7) | 1 (2.0) | |
| | T1c | 51 (48.6) | 14 (28.6) | |
| | T2 | 42 (40.0) | 29 (59.2) | |
| | T3 | 5 (4.7) | 3 (6.0) | |
| | T4 | 0 | 1 (2.0) | |
| N stage | N0 | 53 (50.5) | 21 (42.9) | 0.483 |
| | N1 | 36 (34.3) | 20 (40.7) | |
| | N2 | 12 (11.4) | 4 (8.2) | |
| | N3 | 4 (3.8) | 4 (8.2) | |
| Stage | I | 38 (36.2) | 9 (18.4) | 0.071 |
| | II | 49 (46.7) | 31 (63.3) | |
| | III | 18 (17.1) | 9 (18.4) | |
| Chemotherapy | No | 17 (16.2) | 7 (14.3) | 0.761 |
| | Yes | 88 (83.8) | 42 (85.7) | |
| Hormone therapy | No | 3 (2.9) | 2 (4.1) | 0.654 |
| | Yes | 102 (97.1) | 47 (95.9) | |
| Radiotherapy | No | 52 (49.5) | 23 (46.9) | 0.765 |
| | Yes | 53 (50.5) | 26 (53.1) | |

^{a}SBR: the Scarff-Bloom-Richardson (SBR) grade

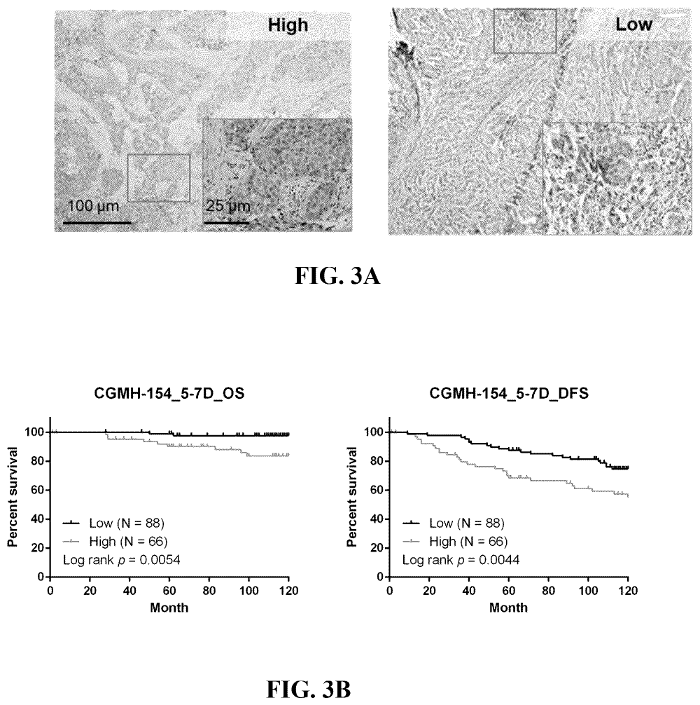

FIGS. 3A and 3B show immunohistochemical analysis of 5-7D staining and its prognostic indications in luminal breast cancer patients. (3A) Representative immunohistochemistry (IHC) images demonstrating 5-7D staining. Magnification: 100× (bottom right: 200×). (3B) Strong immunostaining of 5-7D is associated with decreased overall survival (OS) and disease-free survival (DFS) rates. The IHC Q score was calculated based on the intensity and positive proportion scores. Samples were categorized as Low (Q≤mean) or High (Q>mean).

Paraffin-embedded biopsies were stained with 5-7D, and the IHC results were independently scored by two pathologists using the Q scoring method, which combines the intensity score (ranging from 0 to 3) and the positive proportion score (ranging from 0 to 100). Within our study cohort, we compared the correlation of the 5-7D signal with the IHC results of PKM2 and O-GlcNAc, obtained from the same cohort previously reported by Kuo W L, Tseng L L, Chang C C, Chen C J, Cheng M L, Cheng H H, Wu M J, Chen Y L, Chang R T, Tang H Y et al: Prognostic Significance of O-GlcNAc and PKM2 in Hormone Receptor-Positive and HER2-Nonenriched Breast Cancer. Diagnostics (Basel) 2021, 11(8). Notably, we observed a substantial correlation between 5-7D and anti-O-GlcNAc (r=0.19), while the correlation with anti-PKM2 was low (r<0.1). Subsequently, we stratified the samples into low and high groups based on the 5-7D signal, using the mean value as the threshold. Kaplan-Meier survival analysis demonstrated a significant association between the high 5-7D group and worse overall survival (OS) (p=0.0054) and disease-free survival (DFS) (p=0.00044) (FIGS. 3A and 3B).

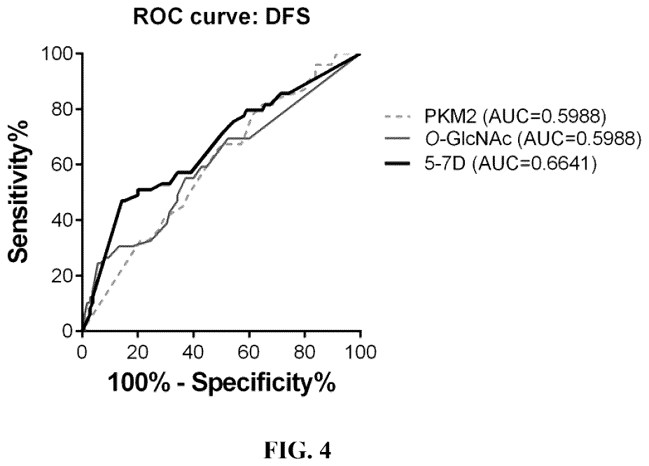

Logistic Regression and Receiver Operating Characteristic (ROC) Curve Analyses. A comparative analysis of logistic regression and receiver operating characteristic (ROC) curves, the 5-7D model exhibited a superior area under the curve (AUC) value of 0.6641, outperforming both PKM2 (AUC=0.5988) and O-GlcNAc (AUC=0.5988) (FIG. 4). These findings collectively reveal the potential of the 5-7D signal as a promising prognostic determinant, granting enhanced precision in predicting clinical trajectories of luminal breast cancer.

In summary, the antibody for predicting breast cancer prognosis of the present invention has the effect on directly identifying the O-GlcNAc protein modification on PKM2 in breast cancer specimens. Prognostic determination can be made with just one staining step, which greatly reduces the cost of testing and is cheaper than previously used gene expression testing methods such as OncotypeDx and MammaPrint. Additionally, in preliminary clinical data analysis, the antibody effectively predicts overall survival (OS) and disease-free survival (DFS) in breast cancer patients. This means that the antibody not only provides an accurate prognostic assessment, but also has a low cost of testing, making it an affordable breast cancer diagnostic tool. Therefore, the antibody of the present invention has the following advantages: 1. Specific recognition of specific molecular marker features: The antibody can specifically recognize specific molecular marker features in breast cancer tissue, such as O-GlcNAc protein modification on PKM2. This specific recognition makes the antibody a reliable tool for predicting the prognosis of breast cancer patients. 2. Highly accurate prognosis prediction: By staining and scoring tissue sections of breast cancer patients, the antibody can provide highly accurate prognosis prediction results. This helps doctors more accurately assess a patient's prognosis and make more informed decisions about individualized treatment. 3. Simplification and cost savings: Compared with traditional multiplex staining methods, the use of this antibody requires only one staining step, thus saving time, labor and material costs. This simplified detection process helps improve efficiency and reduce the cost of detection, making the technology more practical and feasible, and making it less expensive than multigene expression assays such as OncotypeDx and MammaPrint. 4. Application to personalized treatment strategies: The prognostic prediction results of this antibody can provide important reference for personalized treatment strategies for breast cancer patients. By understanding the patient's prognosis, doctors can better select and adjust treatment options to achieve the best clinical results. 5. Potential industrial application value: The application of this technology has potential value in the fields of biomedical testing, clinical diagnosis and pharmaceuticals. Developing related industry products, such as breast cancer diagnostic kits, clinical diagnostic services and drug research and development, can help improve breast cancer diagnosis.

Although the present invention has been described with reference to the preferred embodiments, it will be apparent to those skilled in the art that a variety of modifications and changes in form and detail may be made without departing from the scope of the present invention defined by the appended claims.

What is claimed is:

1. An antibody for predicting breast cancer prognosis, which specifically binds to pyruvate kinase isoenzyme M2 (PKM2), the antibody comprising a plurality of amino acid sequences, wherein each of the plurality of amino acid sequences is complementarity determining region (CDR), and the CDR comprises 5-12 amino acid residues.

2. The antibody according to claim 1, which specifically binds to O-linked β-N-acetylglucosamine (O-GlcNAc) on the PKM2.

3. The antibody according to claim 1, which exhibits an antibody form selected from the group consisting of: single-chain variable fragment (scFv), minibody, nanobody, monoclonal antibody, immunoglobulin G (IgG), immunoglobulin A (IgA), and conjugated form.

4. The antibody according to claim 1, wherein the plurality of amino acid sequences are as follows:
first amino acid sequence, consisting of KSSQSLLYX₁NGK;
second amino acid sequence, consisting of SEQ ID NO:2;
third amino acid sequence, consisting of SEQ ID NO:3;
fourth amino acid sequence, consisting of SEQ ID NO:4;
fifth amino acid sequence, consisting of SEQ ID NO:5; and
sixth amino acid sequence, consisting of MDGX₂YSLFDY;
wherein X₁ is a neutral or hydrophilic amino acid residue, and X₂ is a hydrophobic amino acid residue.

5. The antibody according to claim 4, wherein X₁ is selected from the group consisting of: aspartic acid (D), asparagine (N), glutamine (Q), serine (S), threonine (T), and glutamic acid (E).

6. The antibody according to claim 4, wherein X₂ is selected from the group consisting of: methionine (M), alanine (A), valine (V), isoleucine (I), leucine (L), phenylalanine (F), tyrosine (Y), and tryptophan (W).

7. An isolated nucleic acid, encoding the antibody according to claim 1.

8. A pharmaceutical composition for predicting breast cancer prognosis, comprising the antibody according to claim 1 and a pharmaceutically acceptable carrier.

9. The pharmaceutical composition according to claim 8, wherein the antibody specifically binds to O-linked β-N-acetylglucosamine (O-GlcNAc) on the PKM2.

10. The pharmaceutical composition according to claim 8, wherein the antibody exhibits an antibody form selected from the group consisting of: single-chain variable fragment (scFv), minibody, nanobody, monoclonal antibody, immunoglobulin G (IgG), immunoglobulin A (IgA), and conjugated form.

11. The pharmaceutical composition according to claim 8, wherein the plurality of amino acid sequences are as follows:
first amino acid sequence, consisting of KSSQSLLYX₁NGK;
second amino acid sequence, consisting of SEQ ID NO:2;
third amino acid sequence, consisting of SEQ ID NO:3;
fourth amino acid sequence, consisting of SEQ ID NO:4;
fifth amino acid sequence, consisting of SEQ ID NO:5; and
sixth amino acid sequence, consisting of MDGX₂YSLFDY;
wherein X₁ is a neutral or hydrophilic amino acid residue, and X₂ is a hydrophobic amino acid residue.

12. The pharmaceutical composition according to claim 11, wherein X₁ is selected from the group consisting of: aspartic acid (D), asparagine (N), glutamine (Q), serine (S), threonine (T), and glutamic acid (E).

13. The pharmaceutical composition according to claim 11, wherein X₂ is selected from the group consisting of: methionine (M), alanine (A), valine (V), isoleucine (I), leucine (L), phenylalanine (F), tyrosine (Y), and tryptophan (W).

14. A method for predicting breast cancer prognosis, comprising administering to a sample to be tested the antibody according to claim 1, followed by interpretation of staining results by immunohistochemistry (IHC).

15. The method according to claim 14, wherein the antibody specifically binds to O-linked β-N-acetylglucosamine (O-GlcNAc) on the PKM2.

16. The method according to claim 14, wherein the antibody exhibits an antibody form selected from the group consisting of: single-chain variable fragment (scFv), minibody, nanobody, monoclonal antibody, immunoglobulin G (IgG), immunoglobulin A (IgA), and conjugated form.

17. The method according to claim 14, wherein the plurality of amino acid sequences are as follows:
first amino acid sequence, consisting of KSSQSLLYX₁NGK;
second amino acid sequence, consisting of SEQ ID NO:2;
third amino acid sequence, consisting of SEQ ID NO:3;
fourth amino acid sequence, consisting of SEQ ID NO:4;
fifth amino acid sequence, consisting of SEQ ID NO:5; and
sixth amino acid sequence, consisting of MDGX₂YSLFDY;
wherein X₁ is a neutral or hydrophilic amino acid residue, and X₂ is a hydrophobic amino acid residue.

18. The method according to claim 17, wherein X₁ is selected from the group consisting of: aspartic acid (D), asparagine (N), glutamine (Q), serine (S), threonine (T), and glutamic acid (E).

19. The method according to claim 17, wherein X₂ is selected from the group consisting of: methionine (M), alanine (A), valine (V), isoleucine (I), leucine (L), phenylalanine (F), tyrosine (Y), and tryptophan (W).

20. The method according to claim 14, wherein the sample to be tested is a tissue section.


SEQUENCE LISTING (8 sequences)

SEQ ID NO: 1 - protein, 12 aa, synthetic construct
KSSQSLLYDNGK

SEQ ID NO: 2 - protein, 7 aa, synthetic construct
LVSKLDS

SEQ ID NO: 3 - protein, 9 aa, synthetic construct
VQGTHFPYT

SEQ ID NO: 4 - protein, 5 aa, synthetic construct
SFGMH

SEQ ID NO: 5 - protein, 6 aa, synthetic construct
YISSGS

SEQ ID NO: 6 - protein, 10 aa, synthetic construct
MDGMYSLFDY

SEQ ID NO: 7 - protein, 114 aa, synthetic construct
ELDIQMTQSPLTLSVTIGQPASISYKSSQSLLYDNGKTYLNWLLQRPGQTPKRLIYLVSKLDSGVPVRFTGSGSGTDFTLKISRVEAEDLGVYYCVQGTHFPYTFGGGTKLELK

SEQ ID NO: 8 - protein, 135 aa, synthetic construct
LEVNLEVSGGGLVQPGGSRKLSCAASGFTFSSFGMHWVRQAPERGLEWVAYISSGSSTIYYADTAKGRFTISRDNPKNTLFLQMTSLRSEDTAMYYCARMDGMYSLFDYWGQGTTLTVSSESQSFPNVTSGQAGQ